(19) United States
(12) Patent Application Publication
(10) Pub. No.: US 2019/0216406 A1
(43) Pub. Date: Jul. 18, 2019

(54) WEARABLE DEVICE TO ASSIST COGNITIVE DYSFUNCTION SUFFERER AND METHOD FOR OPERATING THE SAME

(71) Applicant: Robert Polkowski, Boston, MA (US)

(72) Inventor: Robert Polkowski, Boston, MA (US)

(21) Appl. No.: 16/362,194

(22) Filed: Mar. 22, 2019

Related U.S. Application Data
(63) Continuation-in-part of application No. 15/196,943, filed on Jun. 29, 2016.

(51) Int. Cl. A61B 5/00; G06F 3/16; A61B 5/0205; A61B 5/01; A61B 5/11

(52) U.S. Cl. CPC A61B 5/746; G06F 3/167; A61B 5/0002; A61B 5/681; A61B 5/6804; A61B 5/02055; A61B 5/024; A61B 5/0077; A61B 5/11; A61B 5/7405; A61B 5/742; A61B 2560/0252; A61B 5/6831; A61B 5/01

(57) ABSTRACT

A wearable device to assist a sufferer of cognitive dysfunction is disclosed. The wearable device includes a first set of sensors configured to collect data corresponding to a plurality of activities of the sufferer, a second set of sensors configured to monitor surrounding environment, a third set of sensors configured to collect data corresponding to a plurality of activities of one or more surrounding individuals, a transceiver configured to transmit the data corresponding to the first set of sensors, the second set of sensors and the third set of sensors to an external processing device and a notification subsystem configured to activate one or more notifications wherein the one or more notifications comprises one or more active prompts, delivered to the one or more individuals in surrounding of the sufferer, to complete one or more actions associated with the one or more active prompts.

### CROSS-REFERENCE TO RELATED APPLICATION

This application is a continuation-in-part of, and claims the priority benefit of, pending U.S. Non-Provisional application bearing application Ser. No. 15/196,943, filed on Jun. 29, 2016, entitled “Artificial Intelligence Support for Sufferers of Cognitive Dysfunction,” which is hereby incorporated by reference in its entirety.

### BACKGROUND

Embodiments of a present disclosure relates to assistive technology and more particularly to a wearable device to assist cognitive dysfunction sufferer and method for operating the same.

A wearable assistance device is a device which can be worn by a wearer and generate a required information at any time based on the wearer's need. Some of the wearable devices are designed to assist a sufferer of cognitive dysfunction in performing daily activities and hence, reduce need for a care-worker or caregiver. Such wearable devices send reminder messages or guided instructions in response to the sufferer's need for the assistance. Various wearable devices have been utilized for assisting the sufferers of cognitive dysfunction in the activities of the daily living.

Current techniques entail discrete devices that are unable to interact with one another and cannot independently and automatically respond to the daily activities of the sufferer based upon sensor-provided information. Such approaches, therefore, suffer from an inability to recognize the context of the sufferer and deploy correct assistive aids in an adaptive fashion.

Furthermore, some of the known techniques interact with the sufferer by providing a passive prompt, where the passive prompt means a prompt provided to the sufferer, using various electronic devices such as ear-piece or visually via a text prompt on a teleprompter or on special eyeglasses, to help the sufferer to complete the action associated with the prompt which the sufferer is not able to complete due to forgetfulness. However, in some situations the sufferer may not be able to implement the prompt received from the electronic device due to the forgetfulness. In such situations, the problem of incomplete action associated with the prompt remains unsolved by the present techniques.

Hence, there is a need for an improved wearable device to provide assistance to a sufferer of cognitive dysfunction to address the aforementioned issue(s).

### SUMMARY

In accordance with an embodiment of the present disclosure, a wearable device to assist a sufferer of cognitive dysfunction is provided. The wearable device includes a first set of sensors configured to collect data corresponding to a plurality of activities of the sufferer. The wearable device also includes a second set of sensors configured to monitor surrounding environment of the sufferer. The wearable device further includes a third set of sensors configured to collect data corresponding to a plurality of activities of one or more individuals in surroundings of the sufferer. The wearable device further includes a transceiver operatively coupled to the first set of sensors, the second set of sensors and the third set of sensors. The transceiver is configured to transmit the data corresponding to the plurality of activities of the sufferer, monitored surrounding environment of the sufferer and the data corresponding to the plurality of activities of the one or more individuals in surroundings of the sufferer to a processing device to generate a personal data. The wearable device further includes a notification subsystem operatively coupled to the transceiver. The notification subsystem is configured to activate one or more notifications for assistance of the sufferer upon detecting a need based on the plurality of activities of the sufferer, the plurality of activities of one or more individuals in surroundings of the sufferer and one or more patterns recognized by the processing device, wherein the one or more notifications includes one or more active prompts, delivered to the one or more individuals in surrounding of the sufferer, to complete one or more actions associated with the one or more active prompts.

In accordance with another embodiment of the present disclosure, a method for operating the wearable device to assist a sufferer of cognitive dysfunction is provided. The method includes collecting, by a first set of sensors, data corresponding to a plurality of activities of the sufferer. The method also includes monitoring, by a second set of sensors, surrounding environment of the sufferer. The method further includes collecting, by a third set of sensors, data corresponding to a plurality of activities of one or more individuals in surroundings of the sufferer. The method further includes transmitting, by a transceiver, the data corresponding to the plurality of activities of the sufferer, monitored surrounding environment of the sufferer and the data corresponding to the plurality of activities of the one or more individuals in surroundings of the sufferer to a processing device to generate a personal data. The method further includes activating, by a notification subsystem, one or more notifications for assistance of the sufferer upon detecting a need based on the plurality of activities of the sufferer, the plurality of activities of one or more individuals in surroundings of the sufferer and the one or more patterns recognized by the processing device, wherein the one or more notifications includes one or more active prompts, delivered to the one or more individuals in surrounding of the sufferer, to complete one or more actions associated with the one or more active prompts.

To further clarify the advantages and features of the present invention, a more particular description of the invention will follow by reference to specific embodiments thereof, which are illustrated in the appended figures. It is to be appreciated that these figures depict only typical embodiments of the invention and are therefore not to be considered limiting in scope. The invention will be described and explained with additional specificity and detail with the appended figures.

### BRIEF DESCRIPTION OF THE DRAWINGS

The disclosure will be described and explained with additional specificity and detail with the accompanying figures in which:

FIG. 1 is a block diagram representation of a wearable device to assist a sufferer of cognitive dysfunction in accordance with an embodiment of the present disclosure;

FIG. 2 is a block diagram representation of an exemplary wearable device to assist the sufferer of cognitive dysfunction of FIG. 1 in accordance with an embodiment of the present disclosure; and

FIG. 3 is a flow chart representing the steps involved in a method for operating the wearable device to assist the sufferer of cognitive dysfunction of FIG. 1 in accordance with an embodiment of the present disclosure.

Further, those skilled in the art will appreciate that elements in the figures are illustrated for simplicity and may not have necessarily been drawn to scale. Furthermore, in terms of the construction of the device, one or more components of the device may have been represented in the figures by conventional symbols, and the figures may show only those specific details that are pertinent to understanding the embodiments of the present disclosure so as not to obscure the figures with details that will be readily apparent to those skilled in the art having the benefit of the description herein.

### DETAILED DESCRIPTION

For the purpose of promoting an understanding of the principles of the disclosure, reference will now be made to the embodiment illustrated in the figures and specific language will be used to describe them. It will nevertheless be understood that no limitation of the scope of the disclosure is thereby intended. Such alterations and further modifications in the illustrated system, and such further applications of the principles of the disclosure as would normally occur to those skilled in the art are to be construed as being within the scope of the present disclosure.

The terms “comprises”, “comprising”, or any other variations thereof, are intended to cover a non-exclusive inclusion, such that a process or method that comprises a list of steps does not include only those steps but may include other steps not expressly listed or inherent to such a process or method. Similarly, one or more devices or sub-systems or elements or structures or components preceded by “comprises . . . a” does not, without more constraints, preclude the existence of other devices, sub-systems, elements, structures, components, additional devices, additional sub-systems, additional elements, additional structures or additional components. Appearances of the phrase “in an embodiment”, “in another embodiment” and similar language throughout this specification may, but not necessarily do, all refer to the same embodiment.

In the following specification and the claims, reference will be made to a number of terms, which shall be defined to have the following meanings. The singular forms “a”, “an”, and “the” include plural references unless the context clearly dictates otherwise.

Embodiments of the present disclosure relates to a wearable device to assist a sufferer of cognitive dysfunction. The wearable device includes a first set of sensors configured to collect data corresponding to a plurality of activities of the sufferer. The wearable device also includes a second set of sensors configured to monitor surrounding environment of the sufferer. The wearable device further includes a third set of sensors configured to collect data corresponding to a plurality of activities of one or more individuals in surroundings of the sufferer. The wearable device further includes a transceiver operatively coupled to the first set of sensors, the second set of sensors and the third set of sensors. The transceiver is configured to transmit the data corresponding to the plurality of activities of the sufferer, monitored surrounding environment of the sufferer and the data corresponding to the plurality of activities of the one or more individuals in surroundings of the sufferer to a processing device to generate a personal data. The wearable device further includes a notification subsystem operatively coupled to the transceiver. The notification subsystem is configured to activate one or more notifications for assistance of the sufferer upon detecting a need based on a plurality of sensed activities and one or more patterns recognized by the processing device, wherein the one or more notifications comprises one or more active prompts, delivered to the one or more individuals in surrounding of the sufferer, to complete one or more actions associated with the one or more prompts. In the present context, the active prompt is generated for purpose of intimating the one or more individuals in surrounding of the sufferer so that they can assist the sufferer or understand the requirement of the sufferer in a given scenario. The active prompt differs from the passive prompt since the passive prompt is delivered to the sufferer or meant for the sufferer and not for individuals surrounding the sufferer.

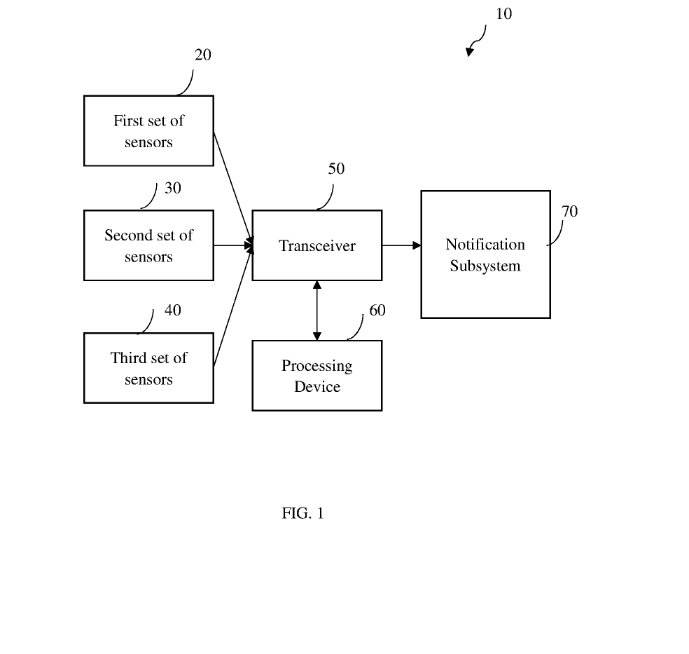

FIG. 1 is a block diagram representation of the wearable device 10 to assist the sufferer of cognitive dysfunction in accordance with an embodiment of the present disclosure. As used herein, “cognitive dysfunction” is defined as a category of neurocognitive disorders that primarily affects cognitive abilities including learning, memory, perception, and problem solving of a person. Here, a wearable device 10 is configured to assist a sufferer of cognitive dysfunction, wherein the wearable device 10 proposes adaptive functionality, assistance and guidance to the sufferer by providing a plurality of actions such as giving guidance or reminders to the user at multiple levels of complexity, taking overrule actions to prevent mistakes or dangerous conditions, or escalating options to warn either the sufferer or nearby higher skilled users such as caregivers. As used herein, “caregiver” encompasses any human other than the sufferer which is in the sufferer's environment or interacts with the sufferer for any reason. Thus, a “caregiver” in accordance with the present invention is not limited to a medical specialist but further includes any human such as a relative, neighbour, and a guest. In one embodiment, the wearable device 10 may include a wrist watch, a strap or a wearable cloth.

The wearable device 10 includes a first set of sensors 20 which is configured to collect data corresponding to a plurality of activities of the sufferer. In one embodiment, the first set of sensors 20 may include a plurality of motion sensors, a plurality of position sensors, and a plurality of biometric sensors. The plurality of motion sensors is configured to collect data corresponding to the type, quantity, and quality of motion-related activities of the sufferer. The plurality of position sensor is configured to collect data corresponding to location and orientation of the wearable, and by reference in certain situations the location of the sufferer in an environment. The plurality of biometric sensors is configured to collect data corresponding to a plurality of biometric parameters of the sufferer such as gait, voice recognition or signature recognition. In another embodiment, the first set of sensors 20 may also include a plurality of skin conductance response sensors, a body or skin temperature sensor and a heart rate sensor. The plurality of skin conductance response sensors and the heart rate sensor are configured to collect data corresponding to skin conductance and heart rate to identify anxiety status of the sufferer. In some embodiments, the IOT standalone sensor is configured to receive information from one or more external devices present in the surrounding of the sufferer. In one embodiment, the plurality of IOT standalone sensors are associated with the wearable device (outside the wearable device) which is configured to set a connection between an external processing device and the plurality of sensors used in the wearable device 10 to communicate the data to the external processing device. In a specific embodiment, the data corresponding to the plurality of activities of the sufferer may include a sufferer's movements, a plurality of interaction events. A routine data, a social life data, and a preference data is collected manually from a plurality of external sources such as a social media platform, a relative or friend.

Furthermore, the wearable device 10 also includes a second set of sensors 30 which is configured to monitor surrounding environment of the sufferer. In one embodiment, the second set of sensors 30 may include a plurality of environmental sensors. In such embodiment, the plurality of environmental sensors may include a temperature sensor, a humidity sensor, a rain sensor, a moisture sensor, a vibration sensor, an illumination sensor and a pressure sensor. The wearable device 10 further includes a third set of sensors 40 which is configured to collect data corresponding to a plurality of activities of one or more individuals in surroundings of the sufferer. In some embodiments, the third set of sensors 40 may include a microphone and an image acquisition device. The microphone is configured to detect speech input of the sufferer as well as surrounding individuals. The image acquisition device may include a still camera or a video camera which is configured to capture an image or video of the sufferer and surrounding individual and surrounding environment. In a specific embodiment, the third set of sensors 40 may be placed directly within the sufferer's environment, and/or may be remote from the sufferer. As used herein, the term “environment” encompasses a physical structure in which the sufferer is located (permanently or periodically) as well as a plurality of entities in that physical structure such as lights, plumbing, ventilation, appliances, humans other than the sufferer that at least periodically visit

Moreover, the wearable device 10 further includes a transceiver 50 which is operatively coupled to the first set of sensors 20, the second set of sensors 30 and the third set of sensors 40. The transceiver 50 is configured to transmit the data corresponding to the plurality of activities of the sufferer, monitored surrounding environment of the sufferer and the data corresponding to the plurality of activities of the one or more individuals in surroundings of the sufferer to a processing device 60 to generate a personal data. In one embodiment, the transceiver 50 may include a beacon transceiver. In some embodiments, the processing device 60 may include a processor located on a local server. In another embodiment, the processing 60 device may include a processor located on a remote server such as a cloud server. In yet another embodiment, the processing device 60 may include a processor located inside the wearable device 10.

In addition, the wearable device 10 includes a notification subsystem 70 which is operatively coupled to the transceiver 50. The notification subsystem 70 is configured to activate one or more notifications for assistance of the sufferer upon detecting a need based on a plurality of sensed activities and one or more patterns recognized by the processing device 60, wherein the one or more notifications includes the one or more active prompts, delivered to the one or more individuals in surrounding of the sufferer or meant for the one or more individuals in surrounding of the sufferer, to complete one or more action associated with the active prompt. The active prompt may include completing the sentence of the sufferer in a conversation with the one or more individuals in surrounding of the sufferer, asking a follow up question meant for the surrounding individual which may help the sufferer in understanding an ongoing conversation or situation, requesting the surrounding individual for any act of assistance to be given to the sufferer, and informing the surrounding individual regarding the mental or physical state of the sufferer. The form of active prompt includes a text notification, an audio notification, a graphic notification, a video notification or any combination thereof.

In one embodiment, the one or more notifications may also include instructions to carry out a task by the sufferer or one or more reminders meant for the sufferer.

In one embodiment, the sentence of the sufferer in a conversation with the one or more individuals in surrounding of the sufferer is completed and communicated to the one or more individuals in surrounding of the sufferer. The assistance in speaking notification is an audio or visual notification which helps sufferer to complete a sentence or recall the name of another individual. The assistance in speaking includes an audio notification which is directly communicated to the one or more individuals in surrounding of the sufferer, when the sufferer is unable to complete the sentence in a conversation with the individuals in surrounding. Hence, the notification subsystem delivers an active prompt (audio notification) to the sufferer as well as the surrounding individuals because the sufferer, in some situations, may be unable to understand the requirement of active prompt. The instruction to carry out a task notification helps a sufferer by sending a notification directing such as “pour the coffee grinds” as part of a procedure such as operating a coffee machine. For example, the one or more reminders may include a reminder for picking up a key when the processing device 60 recognizes that the individual is leaving the house and has not reached for the keys, or a reminder to eat or engage in hygiene activities when the processing device 60 recognizes these activities were not performed as expected. In such embodiment, the one or more notifications may be in a form of at least one of a text notification, an audio notification, a graphic notification or a video notification. In a specific embodiment, the audio notification may be communicated using a speaker associated with the wearable device 10. In one embodiment, the one or more notifications of intervention may be provided to a caregiver to assist the sufferer of cognitive dysfunction when needed. In such embodiment, the caregiver may include but not limited to a medical specialist, a relative, a neighbour or a guest.

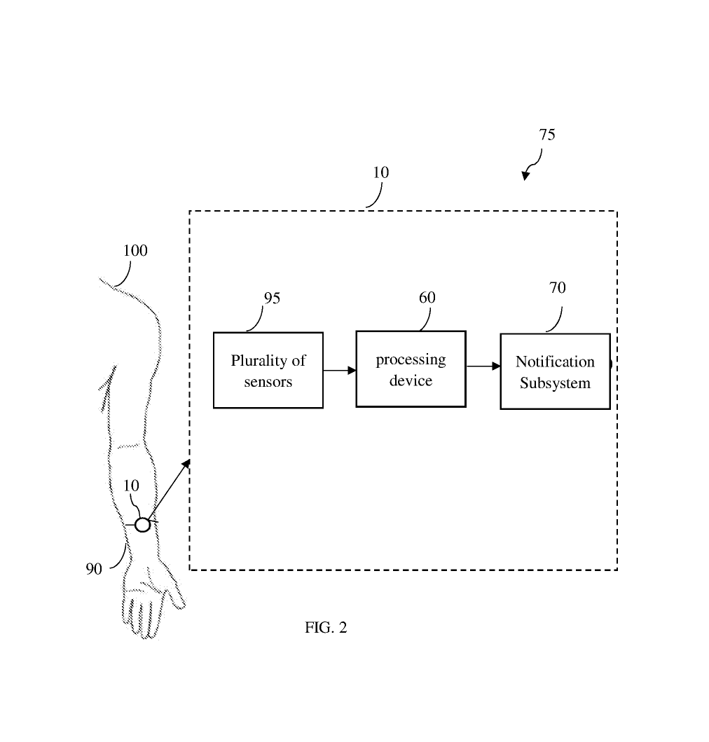

FIG. 2 is a block diagram representation of an exemplary embodiment 75 of the wearable device 10 to assist the sufferer 100 of cognitive dysfunction of FIG. 1 in accordance with an embodiment of the present disclosure. The wearable device 10 provides an assistive technology to promote the independent living of individuals with mild cognitive impairment (MCI) and dementia which is a type of cognitive dysfunction. The assistive technology helps individuals with MCI/dementia maintain independence while improving caregiver quality of life by reducing the burdens of caregiving. The wearable device 10 assists the sufferer 100 of cognitive dysfunction to perform activities of daily living (ADLs) such as managing medications and shopping for groceries, and to continue to perform work-related tasks when an individual experiences lack of focus, confusion or other cognitive difficulties such as completion of sentences during a conversation with surrounding individuals.

Considering a scenario, where the sufferer 100 of dementia has a history of forgetfulness. One day the sufferer 100 is talking to his uncle, who started telling him about a party. During this conversation he asks his uncle “where is the party and . . . ”, but he suddenly stops talking as he is unable to find the right words to express himself; as he struggles to find the words, he also forgets what he was trying to say. The wearable device 10, say a wearable watch which is configured to be coupled on a wrist 90 of the sufferer 100, including a plurality of sensors 95, continuously collects speech data and the data related to his surroundings, including his uncle. The plurality of sensors 95 is substantially similar to first set of sensors 20, the second set of sensors 30 and the third set of sensors 40 of FIG. 1. The plurality of sensors 95 is configured to send such data to a processing device 60 which may be inside or outside the wearable device 10. The processing device 60 analyses the situation by analysing past word combinations, word combinations used in Internet sources and databases, word combinations used by family members or common acquaintances, and/or word combinations used in a place of business, using a personalized support model. The processing device 60 further identifies the current speech pattern and word choice of the sufferer 100, as well as the individual to whom he is talking and his relationship with said person (in this case his uncle). Based on identified patterns, the notification subsystem 70 activates a speech output (active prompt) which is directly communicated to his uncle (because the sufferer in some situations may be unable to understand the requirement of active prompt) saying, “at what time does the party start?”. In such a way the wearable device 10 helped both the sufferer 100 and his uncle learn what the sufferer 100 was trying to convey.

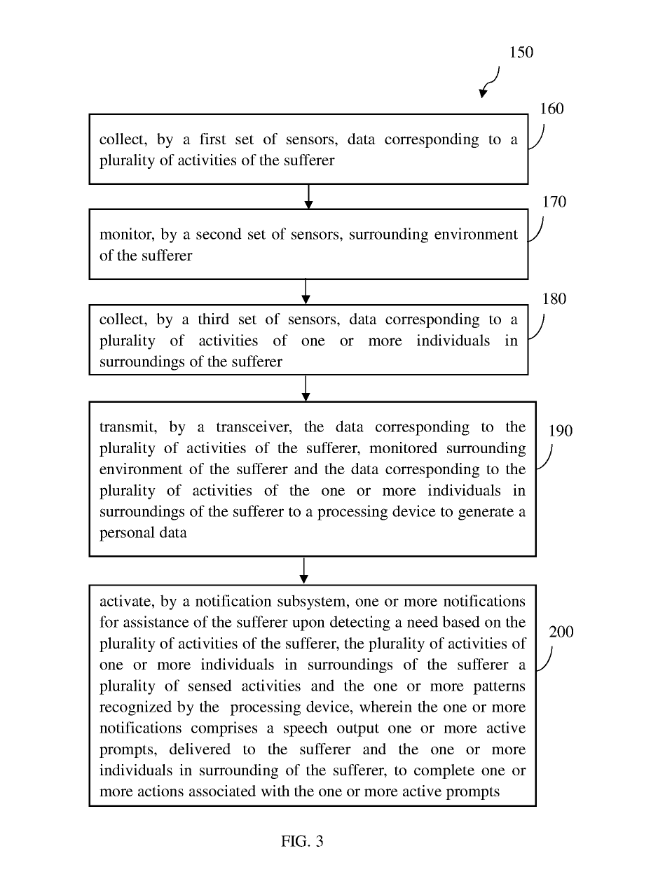

FIG. 3 is a flow chart representing the steps involved in a method 150 for operating the wearable device to assist the sufferer of cognitive dysfunction in accordance with an embodiment of the present disclosure. The method 150 includes collecting, by a first set of sensors, data corresponding to a plurality of activities of the sufferer in step 160. In one embodiment, collecting, by the first set of sensors, the data corresponding to the plurality of activities of the sufferer may include collecting data corresponding to sufferer's movements, a plurality of interaction events. The method 150 also includes monitoring, by a second set of sensors, surrounding environment of the sufferer in step 170. The method 150 further includes collecting, by a third set of sensors, data corresponding to a plurality of activities of one or more individuals in surroundings of the sufferer in step 180.

Furthermore, the method 150 includes transmitting, by a transceiver, the data corresponding to the plurality of activities of the sufferer, monitored surrounding environment of the sufferer and the data corresponding to the plurality of activities of the one or more individuals in surroundings of the sufferer to a processing device to generate a personal data in step 190. The method 150 further includes activating, by a notification subsystem, one or more notifications for assistance of the sufferer upon detecting a need based on the plurality of activities of the sufferer, the plurality of activities of one or more individuals in surroundings of the sufferer and the one or more patterns recognized by the processing device, wherein the one or more notifications comprises one or more active prompts, delivered to the one or more individuals in surrounding of the sufferer, to complete one or more actions associated with the one or more active prompts in step 200. In one embodiment, activating, by the notification subsystem, the one or more notifications for assistance of the sufferer upon detecting the need based on the one or more received patterns may include activating the one or more notifications for instructions to carry out a task, completion of a sentence or one or more reminder upon detecting the need based on the one or more received patterns.

Various embodiments of the wearable device described above enables an efficient system which is configured to constantly collect data from the plurality of sensors and activate one or more notifications to influence the sufferer to perform, resume, correct or abandon the task based on sufferer's current mental state and/or cognitive level.

Furthermore, the wearable device incorporates a highly flexible architecture that is capable of taking input from a wide variety of sensors in a wide variety of settings and send this data to external processing device for mapping the input to a set of events of interest, reasoning about desired responses to those events of interest, and then accomplishing those responses on a wide variety of effectors in a wide variety of settings.

It will be understood by those skilled in the art that the foregoing general description and the following detailed description are exemplary and explanatory of the disclosure and are not intended to be restrictive thereof.

While specific language has been used to describe the disclosure, any limitations arising on account of the same are not intended. As would be apparent to a person skilled in the art, various working modifications may be made to the method in order to implement the inventive concept as taught herein.

The figures and the foregoing description give examples of embodiments. Those skilled in the art will appreciate that one or more of the described elements may well be combined into a single functional element. Alternatively, certain elements may be split into multiple functional elements. Elements from one embodiment may be added to another embodiment. For example, order of processes described herein may be changed and are not limited to the manner described herein. Moreover, the actions of any flow diagram need not be implemented in the order shown; nor do all of the acts need to be necessarily performed. Also, those acts that are not dependent on other acts may be performed in parallel with the other acts. The scope of embodiments is by no means limited by these specific examples.

We claim:

1. A wearable device to assist a sufferer of cognitive dysfunction comprising:
a first set of sensors configured to collect data corresponding to a plurality of activities of the sufferer;
a second set of sensors configured to monitor surrounding environment of the sufferer;
a third set of sensors configured to collect data corresponding to a plurality of activities of one or more individuals in surroundings of the sufferer;
a transceiver operatively coupled to the first set of sensors, the second set of sensors and the third set of sensors, wherein the transceiver is configured to transmit the data corresponding to the plurality of activities of the sufferer, monitored surrounding environment of the sufferer and the data corresponding to the plurality of activities of the one or more individuals in surroundings of the sufferer to a processing device to generate a personal data; and
a notification subsystem operatively coupled to the transceiver and configured to activate one or more notifications for assistance of the sufferer upon detecting a need based on the plurality of activities of the sufferer, the plurality of activities of one or more individuals in surroundings of the sufferer and one or more patterns recognized by the processing device, wherein the one or more notifications comprises one or more active prompts, delivered to the one or more individuals in surrounding of the sufferer, to complete one or more actions associated with the one or more active prompts.

2. The wearable device of claim 1, wherein the wearable device comprises a wrist watch, a strap or a wearable cloth.

3. The wearable device of claim 1, wherein the first set of sensors comprises a plurality of motion sensors, a plurality of position sensors, and a plurality of biometric sensors.

4. The wearable device of claim 1, wherein the first set of sensors comprises a plurality of skin conductance response sensors, a body or skin temperature sensor and a heart rate sensor.

5. The wearable device of claim 1, wherein the second set of sensors comprises a plurality of environmental sensors.

6. The wearable device of claim 5, wherein the plurality of environmental sensors comprises a temperature sensor, a humidity sensor, a rain sensor, a moisture sensor, a vibration sensor, an illumination sensor and a pressure sensor.

7. The wearable device of claim 1, wherein the third set of sensors comprises a microphone and an image acquisition device.

8. The wearable device of claim 1, wherein the data corresponding to the plurality of activities of the sufferer comprises a sufferer's movements and a plurality of interaction events.

9. The wearable device of claim 1, wherein the transceiver comprises a beacon transceiver.

10. The wearable device of claim 1, wherein the one or more actions associated with the one or more active prompt comprises at least one of instructions to carry out a task, sentence completion one or more reminders.

11. The wearable device of claim 1, wherein the one or more active prompts comprises at least one of a text notification, an audio notification, a graphic notification or a video notification.

12. A method comprising:
collecting, by a first set of sensors, data corresponding to a plurality of activities of the sufferer;
monitoring, by a second set of sensors, surrounding environment of the sufferer;
collecting, by a third set of sensors, data corresponding to a plurality of activities of one or more individuals in surroundings of the sufferer;
transmitting, by a transceiver, the data corresponding to the plurality of activities of the sufferer, monitored surrounding environment of the sufferer and the data corresponding to the plurality of activities of the one or more individuals in surroundings of the sufferer to a processing device to generate a personal data; and
activating, by a notification subsystem, one or more notifications for assistance of the sufferer upon detecting a need based on the plurality of activities of the sufferer, the plurality of activities of one or more individuals in surroundings of the sufferer and the one or more patterns recognized by the processing device, wherein the one or more notifications comprises one or more active prompts, delivered to the one or more individuals in surrounding of the sufferer, to complete one or more actions associated with the one or more active prompts

13. The method of claim 12, wherein activating, by the notification subsystem, the one or more notifications for assistance of the sufferer upon detecting the need based on the one or more received patterns comprises activating the one or more notifications for instructions to carry out a task, complete a sentence or one or more reminder upon detecting the need based on the one or more received patterns.

14. The wearable device of claim 12, wherein the one or more active prompts comprises at least one of a text notification, an audio notification, a graphic notification or a video notification.
